(19) United States
(12) Patent Application Publication
(10) Pub. No.: US 2023/0019258 A1
(43) Pub. Date: Jan. 19, 2023

(54) BIR DEZENFEKSIYON SISTEMI

(71) Applicant: TEKNOMASYON MAKINA METAL ELEK. TUR. SAN. TIC. LTD. STI., Bursa (TR)

(72) Inventor: Serdar Alat, Bursa (TR)

(73) Assignee: US Life Tech Inc, Houston, TX (US)

(21) Appl. No.: 16/963,842

(22) PCT Filed: May 20, 2020
(86) PCT No.: PCT/TR2020/050434

§ 371 Date: Jul. 22, 2020

(30) Foreign Application Priority Data
Apr. 9, 2020 (TR) 2020/05714

(51) Int. Cl. A61L 2/00

(52) U.S. Cl. CPC A61L 2/0088; A61L 2202/122; A61L 2202/15; A61L 2202/14

(57) ABSTRACT

Invention relates to a disinfection system which is configured for disinfecting people from the harmful bacteria and viruses that can adhere to the surfaces such as clothes, shoes. More specifically, the present invention relates to a disinfection system which allows efficient usage of the disinfectant fluid by providing maximum effect with the minimum amount of the disinfectant fluid thanks to the spraying unit that can rotate around the human body; can send the particles of disinfection liquid directly to the desired points of the body thus; does not expose the person to excessive disinfectant; moreover, creates hygienic environment for the next disinfection process after each usage by absorbing microorganisms that may be hang in the environment from the previous process thanks to the HEPA filter unit placed in the cabin. Mentioned system is a system that is effective not only on clothes, but also on human skin.

### TECHNICAL FIELD

Invention relates to a disinfection system which is configured for disinfecting people from the harmful bacteria and viruses that can adhere to the surfaces such as clothes, shoes.

More specifically, the present invention relates to a disinfection system which allows efficient usage of the disinfectant fluid by providing maximum effect with the minimum amount of the disinfectant fluid thanks to the spraying unit that can rotate around the human body; can send the particles of excessive disinfectant; moreover, creates hygienic environment for the next disinfection process after each usage by absorbing microorganisms that may be hang in the environment from the previous process thanks to the HEPA (High Efficiency Particulate Arresting) filter unit placed in the cabin. Mentioned system is a system that is effective not only on clothes, but also on human skin.

### PRIOR ART

In the cases such as epidemic, the usage of mask and disinfectants is very important in order to protect against viruses and harmful bacteria. However, using mask and disinfecting hands are not sufficient solutions since these microorganisms (pathogens) which are harmful to human health, can easily attach to the clothes and shoes. As a result of contact with our clothes and shoes, these pathogens begin to spread easily in our body. Especially in the places such as hospital, food production department etc. where it is necessary to minimize microbes, personnel should be disinfected entirely, not only its hand. For this reason, various disinfection systems such as cabin or tunnel have been emerged to provide disinfection of the human body and clothing. The basic principles of all these systems are activating of the nozzles (spray heads) at the different points and/or angles when the person entering the cabin is detected by any sensor and, spraying of the disinfection liquid into cabin as micro particles through these nozzles. Fog cloud formed by the sprayed micro particles reaches pathogens indirectly without creating wetness on the clothes.

Although with the currently used disinfection systems, microorganisms are tried to be eliminated by allowing disinfection liquid to reach the entire outer surface of the person in the cabin, they have various disadvantages. Two most important ones of these are; usage of large amount of disinfectant liquid and being not enough hygienic of the cabin environment for the next disinfection process. Since the currently used disinfection systems are static structures, it is necessary to use a large amount of disinfection fluid to reach the entire surface of the person entering the disinfection cabinet or tunnel. Especially in the tunnel-type disinfection systems, since all the nozzles are activated with the entrance of a person into the cabin, disinfection fluid is sprayed unnecessarily throughout the tunnel. Liquid particles reaches the desired points of the body indirectly, not directly. As a result, disinfectant fluid is not used efficiently and the person is exposed to excessive disinfectant fluid. Worse still, if the previous person entering the disinfection system has a disease such as tuberculosis etc. and breathes or coughs while passing throughout the fog cloud, the person following it has a risk of catching the microbes, viruses or bacteria that have been spread from it. This reveals the require for a solution which allows efficient usage of the disinfectant fluid by providing maximum effect with the minimum amount of the disinfectant fluid and creates hygienic environment for the next disinfection process after each usage by absorbing microorganisms that may be hang in the environment from the previous process.

In the patent document No. CN110448793, a body disinfection device equipped with a monitoring unit is mentioned under the title ‘Human body disinfection device’. All the process while a person is disinfected is monitored by the monitoring unit and recorded by smart terminal adjusted before. Thus, when disinfection problems occur, the video recorded by the monitoring unit can be followed up to its source for finding and solving the problems. The monitoring unit is used for some kind of control purposes. Thus, it is understood whether the person disinfected is severely disinfected. However, in the patent document No. CN110448793, it is not mentioned about a solution which provides maximum effect with the minimum amount of the disinfectant fluid and creates hygienic environment for the next disinfection process.

In the patent document No. CN209316576, under the title ‘Disinfection device for preparing recombinant human epidermal growth factor’, there is a solution for the sterilization studies of the containers especially which are used during the preparation of the human epidermal growth factor (EGF) recombinant. In the conventional method where disinfectant is mostly used to rinse and disinfect the preparation equipment, the liquid on the surface is dried by using a sanitizing fabric. This causes secondary pollution which seriously reduces effect of the disinfection. In order to solve mentioned problem causing pollution in the conventional method, a disinfection device for sterilization of containers used during the preparation of the human epidermal growth factor (EGF) has been disclosed in the document No. CN209316576. However, in this document, it is not mentioned about a solution which sterilizes the human body, provides maximum effect with the minimum amount of the disinfectant fluid and creates hygienic environment for the next disinfection process.

In the patent document No. CN106075709, under the title ‘Closed type intensive care human body disinfection device’ it is mentioned about a device for sterilization of the patient companions of intensive care patients that have low immunity. Here, there are two semi-circular sterilization shells that contain more than one nozzle and are connected to the opposite side surfaces of the cabin my means of multiple telescopic feet. However, in this document, it is not mentioned about a solution which provides maximum effect with the minimum amount of the disinfectant fluid and creates hygienic environment for the next disinfection process.

In the patent document No. KR20130012526, under the title ‘Human disinfection box’, a solution for removal of pathogens adhering to the outer part of the staff which must enter and exit a quarantined area, is mentioned. For providing this, ozone water generator or and electrolytic water generator and an ultraviolet sterilization lamp are configured in the disinfection cabinet. In addition, in order to sterilize and discharge the polluted air after sterilization, and thus to prevent external secondary contamination, the sterilization cabinet contains a large number of photocatalyst filters and a UV sterilization lamp placed on its ceiling. However, in this document, it is not mentioned about a solution which allows efficient usage of the disinfectant fluid by providing maximum effect with the minimum amount of the disinfectant fluid and creates hygienic environment for the next disinfection process by using the HEPA filter.

As a result, the require for a disinfection system which allows efficient usage of the disinfectant fluid by providing maximum effect with the minimum amount of the disinfectant fluid, does not expose the person to excessive disinfectant and creates hygienic environment for the next disinfection process after each usage by absorbing microorganisms that may be hang in the environment from the previous process, caused the present innovative solution to emerge.

### OBJECTIVES AND SHORT DESCRIPTION OF THE INVENTION

This invention presents a disinfection system which allows efficient usage of the disinfectant fluid by providing maximum effect with the minimum amount of the disinfectant fluid thanks to the spraying unit that can rotate around the human body; can send the particles of disinfection liquid directly to the desired points of the body thus; does not expose the person to excessive disinfectant; moreover, creates hygienic environment for the next disinfection process after each usage by absorbing microorganisms that may be hang in the environment from the previous process thanks to the HEPA (High Efficiency Particulate Arresting) filter unit placed in the cabin; is effective not only on clothes, but also on human skin.

A disinfection system which, including disinfection cabin, disinfects the humans from the pathogens that can adhere to the surfaces such as clothes, shoes etc., and comprises a spraying unit including
a rotatable profile with at least one nozzle head on it and
a mechanical rotating unit that enables mentioned rotatable profile to be rotatably connected to the interior of the disinfection cabin,
   in order to prevent the person to be exposed excessive disinfectant by sending the particles of disinfection liquid directly to the desired points of the body, thus to allow efficient usage of the disinfectant fluid by providing maximum effect with the minimum amount of the disinfectant fluid.

Mentioned disinfection system comprises at least one HEPA filter unit including
fan and motor that activate after each usage of mentioned spraying unit and ensure the absorption of the particles remaining in the interior of the cabin and
a HEPA filter that filters pathogens in the absorbed ambient air,
   in order to create hygienic environment for the next disinfection process after each usage by absorbing microorganisms that may be hang in the environment from the previous process.

Said rotatable profile is preferably in the geometries such us inverted U, inverted L etc.

In order to determine when the disinfection process starts; it comprises at least one of the sensors such as weight sensor, thermal sensor, photo sensor etc.

In a preferred embodiment, it comprises weight sensor in its base part in order to determine when the disinfection process starts.

In order to prevent the disinfectant particles to spread around and to ensure the system to be effected minimally by outside weather conditions; it comprises at least one air curtain placed at the entrance and exit of the cabin.

In order to measure the body temperature of person; it comprises at least one non-contact thermometer placed inside or outside of the cabin.

A disinfection system which, including disinfection cabin, disinfects the humans from the pathogens that can adhere to the surfaces such as clothes, shoes etc., and comprises at least one HEPA filter unit including
fan and motor that activate after each usage of mentioned spraying unit and ensure the absorption of the particles remaining in the interior of the cabin and
a HEPA filter that filters pathogens in the absorbed ambient air,
   in order to create hygienic environment for the next disinfection process after each usage by absorbing microorganisms that may be hang in the environment from the previous process.

Mentioned disinfection system comprises a spraying unit including
a rotatable profile with at least one nozzle head on it and
a mechanical rotating unit that enables mentioned rotatable profile to be rotatably connected to the interior of the disinfection cabin,
   in order to prevent the person to be exposed excessive disinfectant by sending the particles of disinfection liquid directly to the desired points of the body, thus to allow efficient usage of the disinfectant fluid by providing maximum effect with the minimum amount of the disinfectant fluid.

### DESCRIPTION OF THE FIGURES

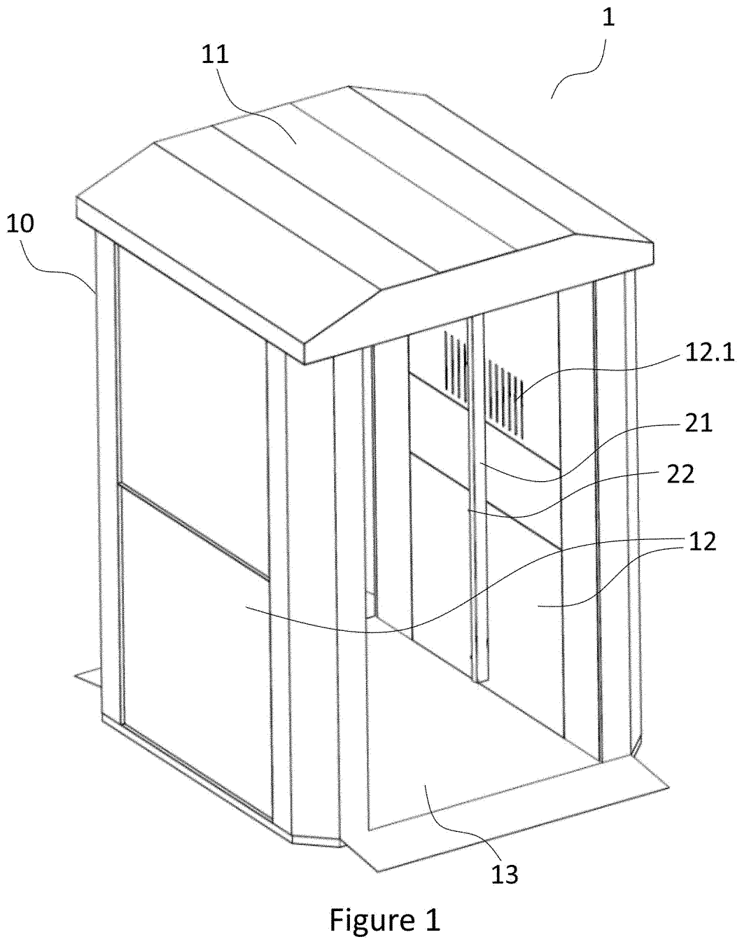

In FIG. 1, a perspective view of the present innovative disinfection system is given.

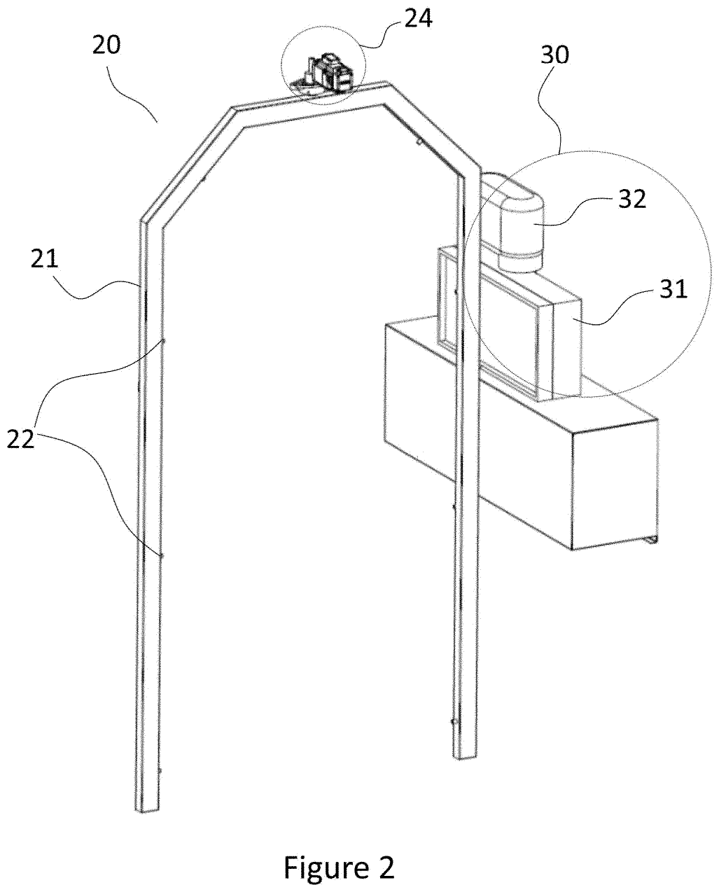

In FIG. 2, a perspective view of the spray unit of the present innovative disinfection system is given.

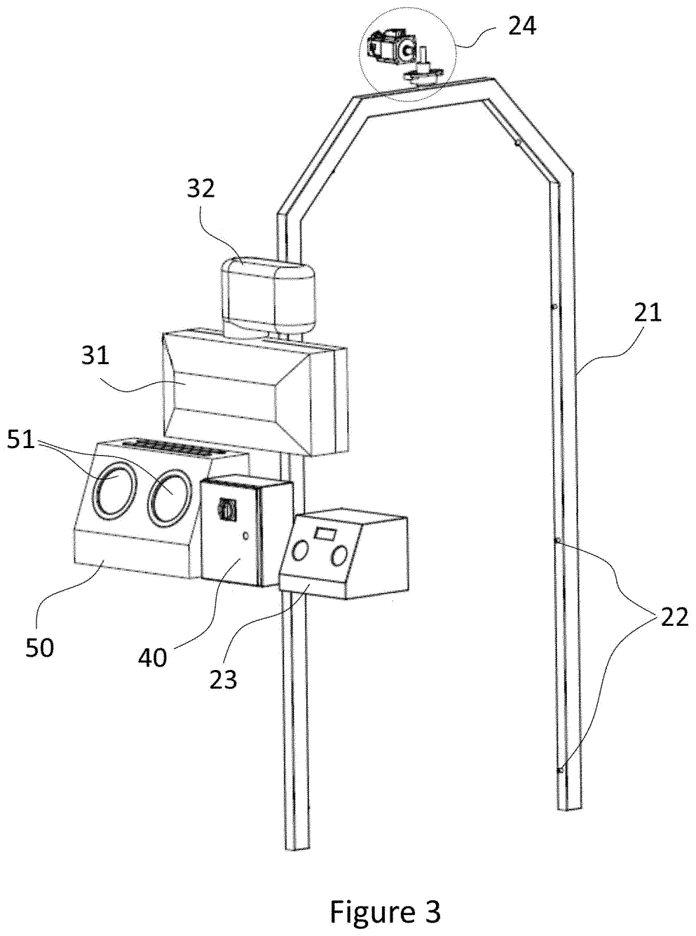

In FIG. 3, another perspective view of the spray unit of the present innovative disinfection system is given.

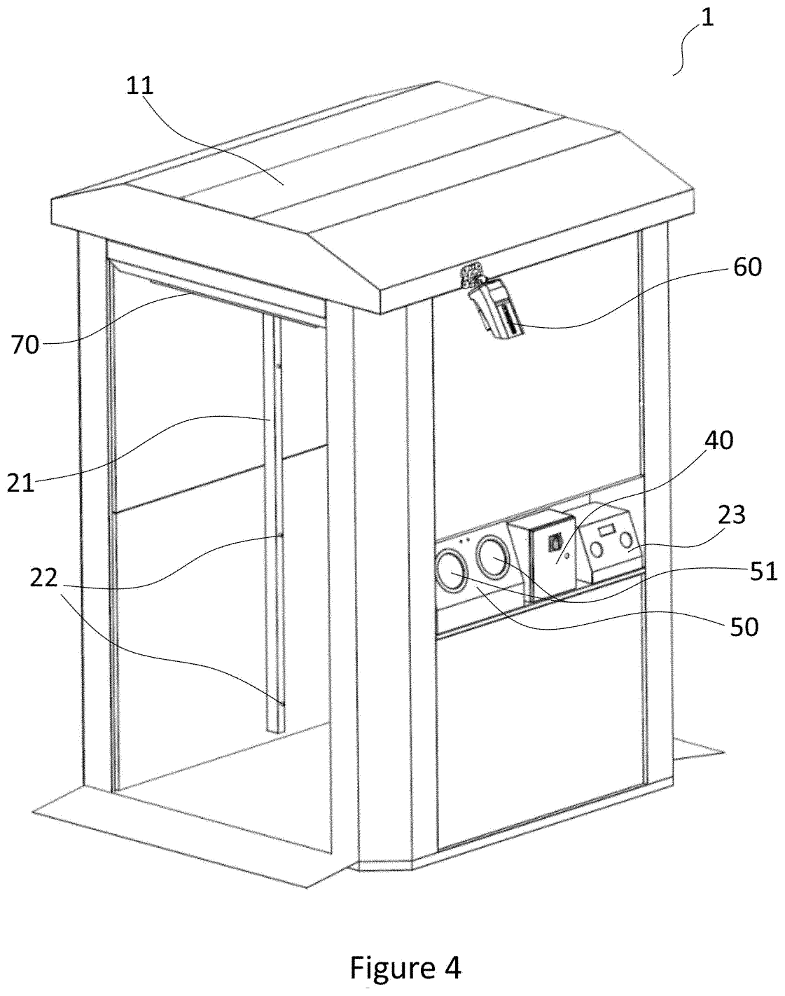

In FIG. 4, a perspective view of the present innovative disinfection system's one embodiment that includes non-contact thermometer and air curtain, is given.

### REFERENCE NUMBERS

1. Disinfection system

10. Disinfection cabin

11. Ceiling

12. Side surface

12.1. Filter cavities

13. Base

20. Spraying unit

21. Rotatable profile

22. Nozzle Heads

23. Control box

24. Mechanical rotation unit

30. HEPA filter unit

31. HEPA filter

32. Fan and Motor

40. Electrical panel

50. Hand disinfector

51. Hand placement cavity

60. Non-contact thermometer

70. Air curtain

### DETAILED DESCRIPTION OF THE INVENTION

Invention relates to a disinfection system (1) which is configured for disinfecting people from the harmful bacteria and viruses that can adhere to the surfaces such as clothes, shoes. More specifically, the present invention relates to a disinfection system (1) which allows efficient usage of the disinfectant fluid by providing maximum effect with the minimum amount of the disinfectant fluid thanks to the spraying unit (20) that can rotate around the human body; can send the particles of disinfection liquid directly to the desired points of the body thus; does not expose the person to excessive disinfectant; moreover, creates hygienic environment for the next disinfection process after each usage by absorbing microorganisms that may be hang in the environment from the previous process thanks to the HEPA filter unit (30) placed in the cabin. Mentioned system is a system that is effective not only on clothes, but also on human skin.

In the disinfection systems that are belong to the known state of the art, since the spraying units are not configured as a rotating structure, more disinfectant liquid should be used for the sprayed micro particles to be reached to the entire outer surface of the person. The spraying unit (20) which is configured as a rotating structure of the present disinfection system (1) allows the spraying process to be carried out along the 360 degree angle around the person. Thus, maximum effect is achieved with the usage of minimum disinfectant fluid, and efficient usage of disinfectant fluid is ensured.

As seen from the figures, the present innovative disinfection system (1) comprises the following components;
a disinfection cabinet (10) configured in the sizes where the human body can enter easily,
a spraying unit (20) which includes
   a rotatable profile (21) with at least one nozzle head (22) on it,
   a mechanical rotating unit (24) that enables mentioned rotatable profile (21) to be rotatably connected to the interior of the disinfection cabin (10) and
   a control box (23),
at least one HEPA filter unit (30) which includes
   fan and motor (32) that activate after each usage of mentioned spraying unit (20) and ensure the absorption of the particles remaining in the interior of the cabin and
   a HEPA filter (31) that filters pathogens in the absorbed ambient air,
thus creates hygienic environment for the next disinfection process,
an electrical panel (40) which provides the electrical energy required for the operation of said units.

In addition, in the preferred embodiment of the invention, there is a hand disinfector (50) placed on the cabin's outer side surface (12) where the person who will enter the disinfection cabin (10) can disinfect its hands firstly. Optionally, the persons can be subjected themselves to a pre-disinfection process by placing their hands within the hand placement cavity (51) on the hand disinfector (50).

The construction of the disinfection system (1) is preferably made of stainless steel (304 quality CrNi) material and is resistant to all weather conditions. The disinfection cabin (10) preferably includes an entrance and an exit. However, in another preferred embodiment of the invention, it is also possible making a cabin configuration where the entrance and the exit are same.

In the preferred embodiment of the invention, the rotatable profile (21) is mounted on the middle of the inner ceiling (11) surface of the disinfection cabin (10) by means of mentioned mechanical rotation unit (24).

The rotatable profile (21) is preferably in the shape inverted U, inverted L etc., and there is at least one nozzle head (22) on rotatable profile's (21) surface facing the cabin environment. In the given embodiment of the invention in figures, there are 8 nozzle heads (22) on the rotatable profile (21) with a certain distance between them. Four of them are placed on one arm of the rotatable profile (21) and remaining four are correspondingly on the other arm. Spraying process is provided by these nozzle heads (22). In said embodiment, these two arms make 180 degrees angle to each other relative to the shaft axis of the mechanical rotation unit (24). In this embodiment, it is sufficient to turn half a turn the rotatable profile (21) in order to make a 360 degree application on the outer surface of the person. For a disinfection system (1) configured with a rotatable profile (21) having 4 arms that make a 90 degree angle to each other relative to the shaft axis of the mechanical rotation unit (24), a quarter turn of the rotatable profile (21) will be sufficient in order to make a 360 degree application on the outer surface of the person. For a disinfection system (1) configured with a rotatable profile (21) having inverted L shape, the rotatable profile (21) must be rotated full turn in order to make a 360 degree application on the outer surface of the person. In the other embodiment of the invention, the rotatable profile (21) can be configured in different geometries.

The person who will enter the disinfection cabin (10) optionally enters the cabin after disinfecting its hands in the hand disinfector (50) placed on the outer side surface (12) of the cabin and steps on the hygiene mat. In this way, the underside of the shoes are also disinfected. In addition, in the preferred embodiment of the invention, the disinfection system (1) is activated with the weight perceived by the weight sensor in the cabin base (13). In other embodiments of the invention, other sensors such as thermal sensors, photo sensors etc. can be used to detect the person entering into the cabin.

The spraying unit (20) activated with activation of the disinfection system (1) sends the disinfection liquid that is brought into the form of fog cloud by using pressure pump, on the person in the cabin by means of nozzle heads (22). Thus, the disinfectant fluid can directly reach to the pathogens on all over the body without creating wetness on the clothes. In this way, exposure of the person to excess disinfectant is prevented. Depending on the geometry of the rotatable profile (21), the profile rotates a quarter turn, half turn, full turn or more for providing 360 degree application to the outer surface of the person. Although not limited with this, standard disinfection time in the cabin is 5 seconds. However, this duration can be set to any value between 3 and 30 seconds.

After the disinfection process (meanly after each usage), the HEPA filter unit (30) is activated and filters the air inside by HEPA filter (31) by ensuring absorption of the microorganisms that may be hang in the environment from the previous process via high pressure fan and motor (32). Thus, a hygienic environment is created for the next disinfection process. On at least one of the inner side surface (12) of the cabin, there are filter cavities (12.1) in which the HEPA filter (31) is placed from the outside. Transmission between the cabin interior space and HEPA filter unit (30) is provided through these filter cavities (12.1).

Unlike disinfection tunnels, the disinfection cabin (10) of the present innovative disinfection system (1) is designed for disinfection of only one person. In this way, the health risks that more than one person can pose for each other are eliminated.

In order to prevent the disinfectant particles to spread around and to ensure the system to be effected minimally by outside weather conditions; at least one air curtain (70) is mounted at the entrance and exit of the cabin, in the preferred embodiments of the invention. In the embodiment where the entrance and the exit are same, it may be sufficient to use one air curtain (70).

In the other preferred embodiment of the invention, there is at least one non-contact thermometer (60) placed inside or outside of the cabin in order to measure the body temperature of person. In the case of detection high fever, an audible/light warning system can be activated.

Biocidal solutions, which does not pose a risk to human health, is intended to be used as a disinfectant liquid in the system. However, it is also possible to use different chemical optionally. In such cases, depending on the preferred disinfectant liquid, the person should be given a face mask before entering the tunnel.

Besides, “ramp systems” designed in accordance with the base (13) height of the cabin are used to eliminate the risks such as stumbling or falling at the entrance and exit of the cabin. Moreover, in the preferred embodiment of the invention, the electricity supply of the disinfection cabin (10) is 220 V or 110 V. However, in order to provide personal safety, the lightening and other electrical systems Inside the cabin are chosen as 12 V or 24 V.

1. A disinfection system (1) which, including disinfection cabin (10), disinfects the humans and/or animals from the pathogens that can adhere to the surfaces such as clothes or shoes, and wherein; in order to prevent the person to be exposed excessive disinfectant by sending the particles of disinfection liquid directly to the desired points of the body, thus to allow efficient usage of the disinfectant fluid by providing maximum effect with the minimum amount of the disinfectant fluid; it comprises a spraying unit (20) including
a rotatable profile (21) with at least one nozzle head (22) on it and
a mechanical rotating unit (24) that enables mentioned rotatable profile (21) to be rotatably connected to the interior of the disinfection cabin (10).

2. A disinfection system (1) according to claim 1, and wherein; in order to create hygienic environment for the next disinfection process after each usage by absorbing microorganisms that may be hang in the environment from the previous process; it comprises at least one HEPA filter unit (30) including
fan and motor (32) that activate after each usage of mentioned spraying unit (20) and ensure the absorption of the particles remaining in the interior of the cabin and
a HEPA filter (31) that filters pathogens in the absorbed ambient air.

3. A disinfection system (1) according claim 1, wherein said rotatable profile (21) is preferably in the geometries such us inverted U or inverted L.

4. A disinfection system (1) according to claim 1, wherein order to determine when the disinfection process starts, it comprises at least one of the sensors such as weight sensor, thermal sensor, or photo sensor.

5. A disinfection system (1) according to claim 4, wherein, in order to determine when the disinfection process starts, it comprises weight sensor in its base (13) part.

6. A disinfection system (1) according to claim 1, wherein in order to prevent the disinfectant particles to spread around and to ensure the system to be effected minimally by outside weather conditions, it comprises at least one air curtain (70) placed at the entrance and exit of the cabin.

7. A disinfection system (1) according to claim 1, wherein in order to measure the body temperature of person, it comprises at least one non-contact thermometer (30) placed inside or outside of the cabin.

8. A disinfection system (1) which, including disinfection cabin (10), disinfects the humans from the pathogens that can adhere to the surfaces such as clothes or shoes, wherein in order to create hygienic environment for the next disinfection process after each usage by absorbing microorganisms that may be hang in the environment from the previous process; it comprises at least one HEPA filter unit (30) including
fan and motor (32) that activate after each usage of mentioned spraying unit (20) and ensure the absorption of the particles remaining in the interior of the cabin and
a HEPA filter (31) that filters pathogens in the absorbed ambient air.

9. A disinfection system (1) according to claim 8, wherein in order to prevent the person to be exposed excessive disinfectant by sending the particles of disinfection liquid directly to the desired points of the body, thus to allow efficient usage of the disinfectant fluid by providing maximum effect with the minimum amount of the disinfectant fluid, it comprises a spraying unit (20) including
a rotatable profile (21) with at least one nozzle head (22) on it and
a mechanical rotating unit (24) that enables mentioned rotatable profile (21) to be rotatably connected to the interior of the disinfection cabin (10).

10. A disinfection system (1) according to claim 2, wherein said rotatable profile (21) is preferably in the geometries such us inverted U or inverted L.

11. A disinfection system (1) according to claim 2, wherein, in order to determine when the disinfection process starts, it comprises at least one of the sensors such as weight sensor, thermal sensor, or photo sensor.

12. A disinfection system (1) according to claim 3, wherein, in order to determine when the disinfection process starts, it comprises at least one of the sensors such as weight sensor, thermal sensor, or photo sensor.

13. A disinfection system (1) according to claim 2, wherein in order to prevent the disinfectant particles to spread around and to ensure the system to be effected minimally by outside weather conditions, it comprises at least one air curtain (70) placed at the entrance and exit of the cabin.

14. A disinfection system (1) according to claim 3, wherein in order to prevent the disinfectant particles to spread around and to ensure the system to be effected minimally by outside weather conditions, it comprises at least one air curtain (70) placed at the entrance and exit of the cabin.

15. A disinfection system (1) according to claim 4, wherein in order to prevent the disinfectant particles to spread around and to ensure the system to be effected minimally by outside weather conditions, it comprises at least one air curtain (70) placed at the entrance and exit of the cabin.

16. A disinfection system (1) according to claim 5, wherein in order to prevent the disinfectant particles to spread around and to ensure the system to be effected minimally by outside weather conditions, it comprises at least one air curtain (70) placed at the entrance and exit of the cabin.

17. A disinfection system (1) according to claim 2, wherein in order to measure the body temperature of person, it comprises at least one non-contact thermometer (30) placed inside or outside of the cabin.

18. A disinfection system (1) according to claim 3, wherein in order to measure the body temperature of person, it comprises at least one non-contact thermometer (30) placed inside or outside of the cabin.

19. A disinfection system (1) according to claim 4, wherein in order to measure the body temperature of person, it comprises at least one non-contact thermometer (30) placed inside or outside of the cabin.

20. A disinfection system (1) according to claim 5, wherein in order to measure the body temperature of person, it comprises at least one non-contact thermometer (30) placed inside or outside of the cabin.
